(19) United States
(12) Patent Application Publication
(10) Pub. No.: US 2013/0047981 A1
(43) Pub. Date: Feb. 28, 2013

(54) DISPENSER WITH A RATTLE

(71) Applicant: Raymond John Bacon, Steep (GB)

(72) Inventor: Raymond John Bacon, Steep (GB)

(21) Appl. No.: 13/582,173

(22) PCT Filed: Mar. 4, 2011
(86) PCT No.: PCT/GB2011/000311

§ 371 Date: Nov. 14, 2012

(30) Foreign Application Priority Data
Mar. 4, 2010 (GB) 1003581.4

(51) Int. Cl. A61M 11/04

(52) U.S. Cl. 128/200.23

(57) ABSTRACT

A metered dose inhaler (1) has a pressed aluminium vessel, to which is crimped at a neck (2) a valve housing (3) having a dispensing spout (4). Within the vessel is a piston (5), urged by a spring 6 into contact with the medicament liquid (7). A bearing ball (8) is included in the medicament liquid. As the medicament liquid is dispensed in successive doses, the piston is kept in contact with the liquid (7) by the spring (6), with an edge seal (9) ensuring that the liquid does not pass to the spring side of the piston. As the medicament liquid is dispensed, the piston begins to restrict the movement of the ball, until it is scarcely able to move longitudinally of the vessel. This change is discernible as a change in noise of the rattle. Thus the user can know whether there is plenty of medicament liquid left and when he needs to use a new inhaler.

The present invention relates to a pressurised-liquid, dispensing container.

Dispensers such as metered dose inhalers enable users to self administer doses of inhaled medication for relief and control of diseases such as asthma.

There is an increasing requirement for these inhalers to be fitted with counters, to count the number of doses dispensed, so that users know when their devices are nearly empty. However, since counters are either intricate mechanical devices or electronic devices, they add considerably to the cost of the inhaler to which they are fitted.

Metered dose inhalers use a pressurised-liquid, dispensing container of the following type, “the type defined”, namely a pressurised-liquid, dispensing container comprising:

a valve for dispensing the liquid from the container; and

a vessel containing the liquid under pressure, the vessel having:
a valve end to which the valve is attached,
a sidewall having an at least partially cylindrical shape and
an end wall.

The object of the present invention is to provide an improved means of indicating the quantity of liquid, typically medication, remaining in a container of this type.

According to the invention there is provided a pressurised-liquid, dispensing container of the type defined, the container containing a pressurised liquid and a propellant gas, the container including:
at least one rattle provided to be movable within the vessel for production of a noise when the container is acted on to induce the noise and
at least one means for restricting movement (“movement restricter”) of the rattle to one side of the movement restrictor or a part thereof,
   the arrangement being such that freedom of movement of the rattle is influenced not only by the movement restrictor, but also by the extent if any to which space in the vessel on the rattle's side of the movement restrictor or a part thereof is no longer occupied by liquid dispensed from the container, whereby noise produced by the rattle varies in accordance with the amount of liquid in the container, at least as the liquid approaches final depletion towards the container being empty.

As used herein, the term “rattle” means an object which produces its noise when the container is acted on as aforesaid. Normally the container and/or a dispenser having the container will have instructions to shake, tap or otherwise act on the container to induce the noise for estimation of the amount of liquid in the container, at least as the liquid approaches final depletion to the container being empty, the instructions including which of the end wall and the valve end of the vessel should be uppermost when the rattle is excited to induce noise. The instructions can be provided with the container or dispenser or printed on the container or dispenser.

The or each rattle can be any suitable object which can move within the vessel and produce a noise when the container is acted upon to induce a noise. Normally the rattle will be small with respect to the vessel. Again, preferably it is solid, although it can be envisaged to be hollow. Examples of suitable objects include a ball, pellet, cone, wedge, cylinder, pyramid, torus, cube, prism, tetrahedron, octahedron, dodecahedron, icosahedron or fragment thereof, or a wire based object, such as a spring, folded wire or other arrangement of wire.

Normally the liquid is a medicament liquid having:

a medicament dissolved in a solvent or

a medicament dispersed in a liquid as a colloid or emulsion or

a medicament suspended in a liquid as a suspension.

The vessel can be any suitable type of vessel to hold medicament liquid. Preferably it is an aluminium vessel.

In certain embodiments, the liquid is one having a vapour phase at room temperature acting as a propellant gas pressuring the liquid for its dispensing. In others, a propellant gas is included which is different from the liquid for pressurising the liquid for its dispensing.

In certain embodiments, the or one of the rattles is buoyant in the liquid. In these embodiments, the buoyant rattle can be between 15% & 50% and preferably 20% & 30% of the cross-sectional dimension of the vessel.

In other embodiments, the or one of the rattles is non-buoyant in the liquid. In these embodiments, the non-buoyant rattle can be between 5% & 15% and preferably 7.5% & 12.5% of the cross-sectional dimension of the vessel. In these embodiments, the preferred rattle is a solid bearing ball.

Advantageously, the rattle will be composed of an inert material, such that it does not react with the container's contents.

Whilst most embodiments use one rattle, more than one can be provided, conceivably up to five or even ten or more. Furthermore, the types of rattles used can be the same (e.g. two balls) or different (e.g. one ball and one spring).

The movement restrictor can be a physical restriction limiting the rattle to part only of the internal space of the container, such that as the level of the medicament liquid within the container sinks below a threshold, the rattle is able to move within the propellant gas, i.e. in space no longer occupied by liquid. Such a movement restrictor can be:
at least one indent in the sidewall of the vessel, restricting movement of the rattle past the indent; or
an element fixed into the vessel, restricting movement of the rattle past the element; or
a link inside the vessel attaching the rattle to the vessel.

In one embodiment, the container includes two rattles and two movement restrictors, the restrictors limiting their rattles to movement along different lengths of the vessel, whereby on continued depletion of the liquid with dispensing, first one rattle and then the other is able to move in space no longer occupied by liquid.

In other embodiments, the movement restrictor can be arranged to be movable so as to restrict the rattle progressively as the medicament is used. In such arrangement, the restriction means can be a separator separating the propellant gas from the medicament liquid and limiting movement of the rattle within the medicament liquid, whereby as the medicament liquid is consumed by successive dispensing, the rattle is progressively restricted in its noise-producing movement.

The separator can be a piston with a spring urging the piston towards the medicament in its carrier.

Alternatively, the separator can be a diaphragm separating propellant gas from the medicament in its carrier.

Where a separator is provided two rattles can be provided, with one being larger than the other, whereby the large one can become held by the restriction means before depletion with the other still movable, where the noise of the smaller one only indicates a small quantity only of contained liquid remaining.

It can also be envisaged that the separator can act to free the rattle progressively as the medicament liquid is dispensed.

The physical restriction can be a cage arranged within the container or one of more indents in a side wall of the container or indeed a link attaching the rattle to an end of the container.

Various alternative configurations of the cage are envisaged:

with the cage limiting the rattle from reaching one end only of the vessel,

with the cage limiting the rattle from reaching either end of the vessel,

with the cage located by crimps in the sidewall of the vessel.

In a somewhat different embodiment:
the rattle is buoyant and
the movement restrictor is a feature of the shape of the rattle providing the rattle with length such that in an upright orientation of the container, the buoyancy of the rattle holds it against the end of the container which is upper most until the liquid approaches final depletion.

For all embodiments, two discrete alternatives are possible, i.e. that:
the or each rattle is arranged on the end wall side of the or each restrictor or part thereof for indication of depletion with the valve end of the vessel upper most; or that
the or each rattle is arranged on the valve end side of the or each restrictor or part thereof for indication of depletion with the end wall of the vessel upper most.

Normally, the container will be in combination with instructions to shake, tap or otherwise act on the container to induce the noise at least as the liquid approaches final depletion to the container being empty, the instructions including which of the end wall and the valve end of the vessel should be uppermost when the rattle is excited to induce noise. The instructions can be printed on a label on the container or on a dispenser having the container.

To act on the container to induce the noise, it can be shaken, juddered, rocked, tapped etc. This action will induce movement of the rattle, if it is free to move within the vessel, causing it to impact with the walls of the vessel or means for restricting its movement. This impact will make a noise. If the container is shaken, the resultant succession of short noises or sounds are likely to be heard as a rattling of the rattle.

Where, as in some embodiments, the rattle is progressively freed with use of the medicament liquid, the movement of the rattle is restricted and on impact with the walls of the vessel it will produce little noise. The presence of liquid around the rattle is liable to dampen the impact. When the rattle is able to move more freely with dispensing of liquid, it will be able to impact harder on the vessel walls and produce a discernibly different sound. This is particularly the case when the rattle is free to move in the propellant gas, i.e. in space no longer occupied by liquid.

Thus, as the freedom of the rattle can be correlated with the amount of liquid within the vessel, acting upon the container to produce a noise (e.g. shaking the container) can determine approximately how much liquid is remaining by the noise made. The user can know whether or not there is liquid left in the container simply by shaking it. The extent of use of the liquid is indicated by the noise produced by the rattle.

To help understanding of the invention, various specific embodiments thereof will now be described by way of example and with reference to the accompanying drawings, in which:

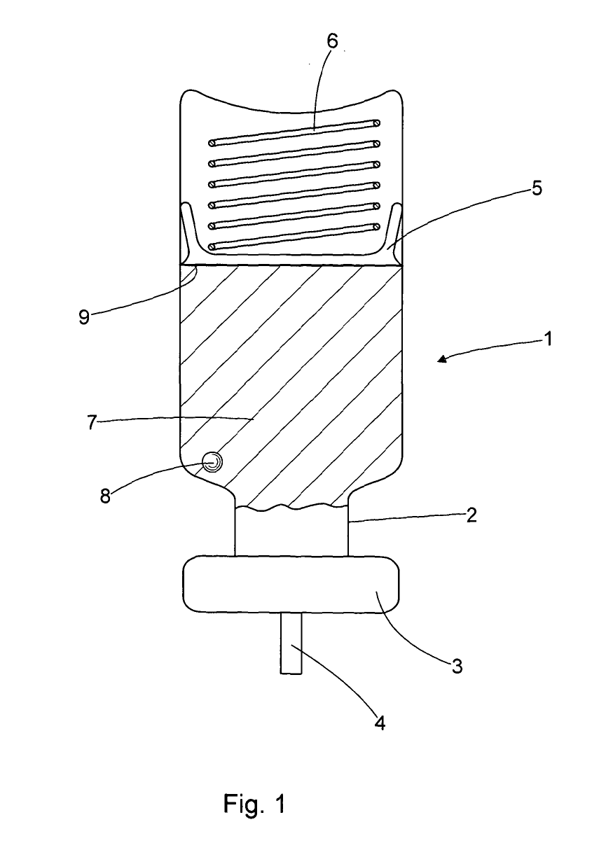

FIG. 1 is a partially sectioned medicament dispenser in accordance with the invention, having a moving piston;

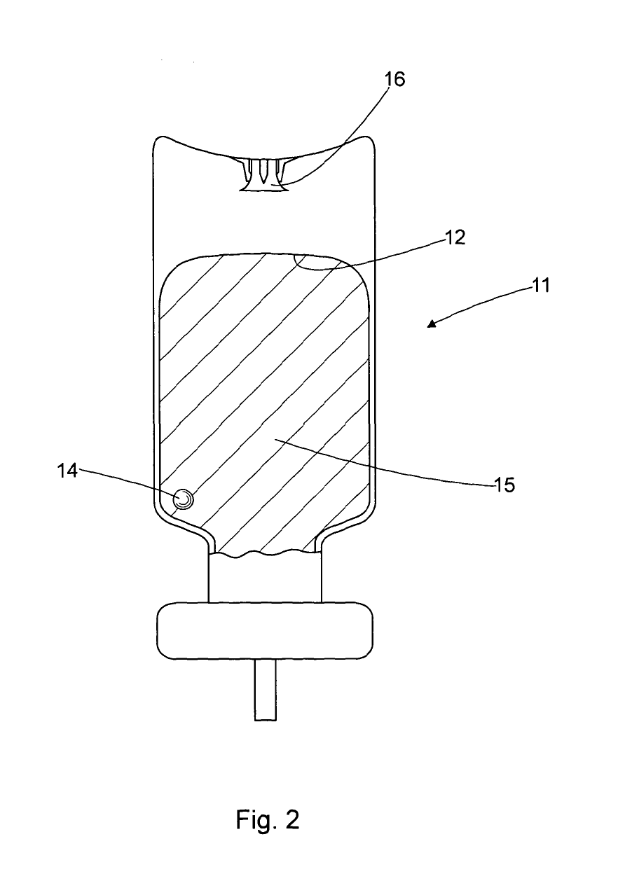

FIG. 2 is a similar view of another dispenser of the invention, having a moving diaphragm;

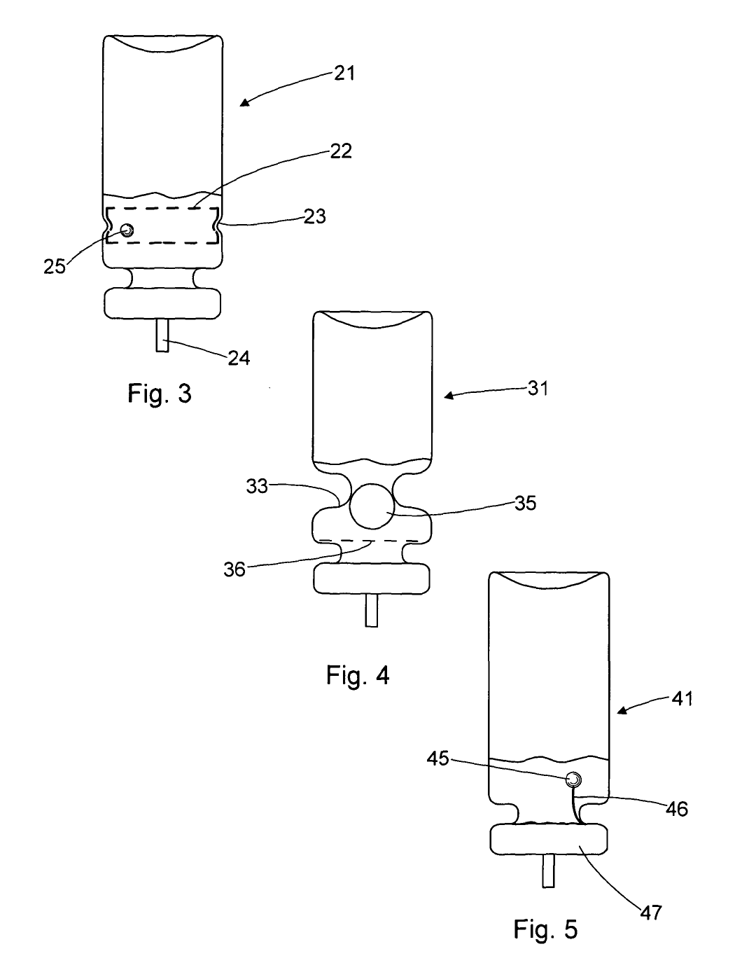

FIG. 3 is a third dispenser of the invention having a ball cage;

FIG. 4 is a fourth dispenser of the invention having a crimp restricted ball;

FIG. 5 is a fifth dispenser of the invention having a thread restricted ball; and

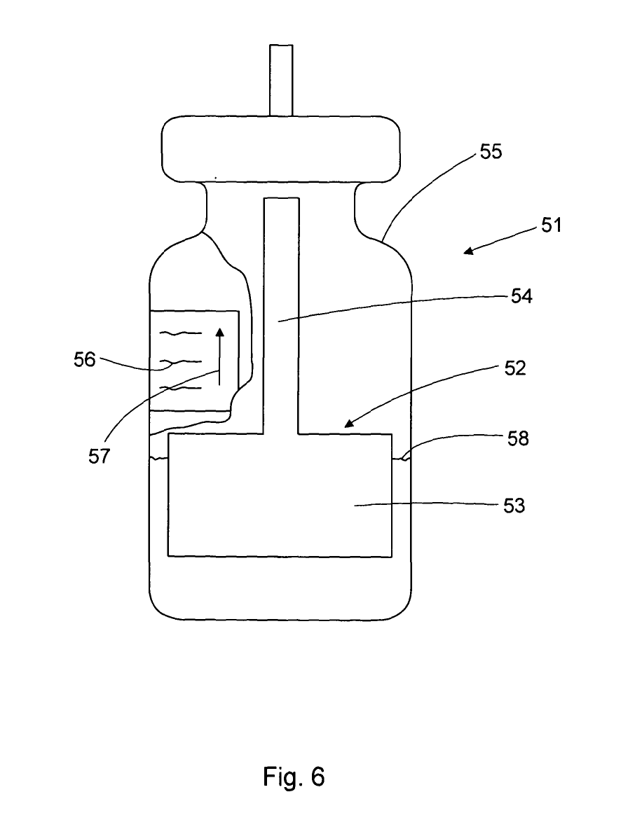

FIG. 6 is a sixth dispenser of the invention having a longer buoyant rattle.

Referring to first to FIG. 1, a container 1 of a metered dose inhaler has a pressed aluminium vessel, to which is crimped at a neck 2 a valve housing 3 having a dispensing spout 4.

Within the vessel is a piston 5, urged by a spring 6 into contact with the medicament liquid 7. A bearing ball 8 is included in the medicament liquid.

As the medicament liquid is dispensed in successive doses, the piston is kept in contact with the liquid 7 by the spring 6, with an edge seal 9 ensuring that the liquid does not pass to the spring side of the piston.

Initially there is no restriction on movement of the ball 8 if the inhaler is shaken, causing the ball to rattle. As the medicament liquid is dispensed, the piston begins to restrict the movement of the ball, until it is scarcely able to move longitudinally of the vessel. This change is discernible as a change in noise of the rattle. Thus the user can know whether there is plenty of medicament liquid left and when he needs to use a new inhaler.

It is envisaged that the seal might not be needed if the piston were of strongly hydrophobic or oleophobic material according to the nature of the medicament material. Further, it may be that the spring can be dispensed with if the piston is of a material to remain preferentially in contact with the medicament.

It is also envisaged that the spring could have a free length such that it is free to rattle between the piston and the end wall of the vessel when the piston has reached a position in which the medicament liquid is nearly used up. In this alternative, the spring performs the noise producing function. Provision of two springs can also be envisaged, with one acting to move the piston and the second having a limited free length. When the piston has moved more than the free length, the second spring expands no further and as such becomes free to rattle as the piston moves further.

Further it is also envisaged that two different sized rattle balls could be provided, possibly of different materials. Initially rattling of the container will produce a dull rattle of a lighter larger ball and a sharper rattle of the heavier smaller ball. As dispensing continues, the reassuring thump of the larger ball ceases as it is captivated between the piston and the valve end of the vessel; this leaves the shriller alarm of the smaller ball indicating that depletion is nearly complete.

Referring to FIG. 2, the medicament in this container 11 is contained within a diaphragm 12 inside an aluminium vessel. A ball 14 is contained with the medicament liquid 15 and a propellant gas introduced via a valve 16 pressurises the medicament liquid via the diaphragm. Again shaking of the inhaler indicates the extent of its use in that as the medicament liquid is used, the ball's freedom to move and produce noise is progressively restricted. The diaphragm can be a plain bag or a bellows diaphragm or a rolling diaphragm.

As an alternative to the diaphragm, a sealed piston with propellant gas as opposed to a spring urging the piston against the medicament liquid can be envisaged.

Whilst in the two embodiments and alternatives just described the piston and diaphragm move to a position increasingly restricting the freedom of the ball to make noise on dispensing (except for the possibility of the spring becoming free to rattle on dispensing); in the three embodiments described below fixed—or at least tightly limiting—means are provided for restricting movement of a noise producing ball, which is freed to make noise when the medicament liquid level falls through dispensing to allow the ball to move more freely in propellant gas or in the space that is no longer fully filled with medicament liquid.

In FIG. 3, the container 21 has a thick disc shaped cage 22 formed of perforate sheet arranged across the vessel of the container close to its end having a spout 24. The cage is held in position by crimps 23 in the vessel sidewall. Within the cage a rattle ball 25 is captive. Whilst the container is full of medicament liquid, or mostly so, tapping the side of the container, or otherwise shaking it with the spout down will cause the ball to strike the side of the cage and the sidewall of the container in the region of the crimps. However, the impact will be dampened by the presence of the liquid. When the liquid level falls below the level of the cage, tapping will cause the ball to impact the sidewall more firmly in the absence of damping from the medicament liquid. The fact that the medicament is mostly dispensed will be evident from the noise generated.

The cage is shown in FIG. 3 to be two-sided, restraining the rattle ball from reaching either end of the vessel. It could be single sided and allow the ball to reach whichever side is appropriate for indicating imminent depletion of liquid, with the missing side lowermost. It will be appreciated that if the container is to be rattled with the spout uppermost, the cage should be nearer the end wall as opposed to nearer the valve end as shown in FIG. 3.

In a non-illustrated embodiment, a second cage and a second rattle ball is provided spaced longitudinally in the vessel from the first. At first with the container full, neither ball can rattle audibly. Then as the level falls to the first cage, its ball can be rattled and heard. As the liquid level falls further with depletion, the second rattle ball can be heard, indicating imminent complete depletion.

The container 31 of FIG. 4 differs in that the ball 35 is buoyant and restrained close to the spout end by more substantial crimps 33. When the container is tapped with plenty of medicament left, the ball will float in the liquid against the crimps and negligible noise will be produced. However, when the liquid level falls below the crimps, the ball will be free to move and will impact with the side wall again indicating with the impact noise that the container is approaching empty. In a variant, a perforated restrictor 36 is provided across the vessel of the container and below the crimps close to the valve end, so that the ball remains above liquid level when the liquid level falls below the perforated restrictor. This has the advantage that two different sounds are produced, one with the ball able to move but still floating, the second with the ball completely out of the liquid.

A third alternative is shown in FIG. 5. The sidewall of the vessel of the container 41 is un-crimped. A buoyant ball 45 is restrained by a strip or thread 46, fast in the crimping of the valve housing 47. Again when the container is full the ball's movement is damped. When the liquid level falls, the ball can impact the vessel's wall without damping and make empty indicating noise. In a variant of this embodiment, using a non-buoyant ball, the dispenser is tested in the inverted position, i.e. spout up. Initially this causes the ball to be in liquid and then as the dispenser gets close to empty the ball is in empty space and as such is able to hit the side wall of the vessel in an un-damped fashion.

It is also envisaged that two balls, suspended at different depths and separated by an internal wall, could be provided so that as the liquid level drops first one ball impacts the side wall and then with further liquid depletion the two balls can hit the side walls, giving a recognisably different noise.

A somewhat different embodiment is shown in FIG. 6, in which a container 51 is provided with a buoyant rattle 52 is provided. It is essentially Tee shaped in side view, with a main float 53 and a thin extension 54 on the valve end side of the float. Whilst there remains a substantial proportion of the liquid in the container, with the valve end 55 up—the orientation instructed by instructions 56, including an orientation arrow 57, adhered onto the container—the float presses the extension against the valve end of container. Tapping of the container will not cause appreciable movement of the float, whose movement is in effect restricted by the extension and the fact that the main float is not much smaller in diameter than that of the bore of the vessel. Once dispensing has continued to the point where the extension is no longer buoyantly urged against the valve end, tapping of the container will cause the float to rattle against the side of the vessel in an audible manner. As dispensing continues, the float will be floating well up on the surface 58 of the liquid and tapping produces a more easily discernible rattle. This is particularly the case where the tip of the extension is able to strike the sidewall of the vessel.

In an non-illustrated alternative, with the main float longer & narrower and the extension shorter, the container can be tested with the end wall uppermost. In this case, the buoyant rattle float will float up against the end wall until much of the contents has been dispensed. Then when it is floating freely it will be able to rattle in a dulled manner, with the liquid dampening the rattle. On continued dispensing, as the liquid nears complete depletion, the extension will hold the float up and it will be able to rattle against the sidewall with no dampening at all at the end of the float remote from the down-most valve end of the container.

So that the container does not have to be removed from the dispenser body that it is intended to be used with, the instructions can be printed on a label adhered to the dispenser body.

Finally, it can be envisaged that the change in noise of the rattle can be detected externally to the container by an electrical transducer, enabling an electronic indication of medicament liquid presence.

Normally in the above described embodiments, the amount of medicament liquid in the container will vary in accordance with the amount already dispensed from it. Thus the noise produced will normally indicate the amount remaining and thus the amount dispensed. However, occasionally a particular dispensing device's valve will leak. In this case the amount remaining varies without dispensing and a conventional dose counter would not indicate the amount remaining. However, advantageously in the above embodiments, the noise produced by the rattle will vary with both dispensing usage and leakage.

1-31. (canceled)

32. A dispensing container of the type defined, the container containing a pressurised liquid and a propellant gas, the container including:
at least one rattle provided to be movable within the vessel for production of a noise when the container is acted on to induce the noise and
at least one means for restricting movement (“movement restrictor”) of the rattle to one side of the movement restrictor or a part thereof,
the arrangement being such that freedom of movement of the rattle is influenced by the movement restrictor and/or by the extent to which space in the vessel on the rattle's side of the movement restrictor or a part thereof is no longer occupied by liquid dispensed from the container, whereby noise produced by the rattle varies in accordance with the amount of liquid in the container, at least as the liquid approaches final depletion towards the container being empty.

33. A pressurised-liquid, dispensing container as claimed in claim 32, wherein the movement restrictor is a physical restriction limiting the rattle to part only of the internal space of the vessel, such as at least one indent in the sidewall of the vessel which restricts movement of the rattle past the indent, an element fixed into the vessel which restricts movement of the rattle past the element, a cage arranged within the vessel or a link inside the vessel which attaches the rattle to the vessel, so that as the level of the liquid within the vessel sinks below a threshold, the rattle is able to move within the propellant gas.

34. A pressurised-liquid, dispensing container as claimed in claim 32, including two rattles and two movement restrictors, the restrictors limiting their rattles to movement along different lengths of the vessel, whereby on continued depletion of the liquid with dispensing, first one rattle and then the other is able to move in space no longer occupied by liquid.

35. A pressurised-liquid, dispensing container as claimed in claim 32, wherein the movement restrictor is arranged to be movable inside the vessel, so as to change the freedom of movement of the rattle progressively as the liquid is dispensed.

36. A pressurised-liquid, dispensing container as claimed in claim 35, wherein the restriction means is a separator separating the propellant gas from the liquid and limiting movement of the rattle within the liquid, whereby as the liquid is consumed by successive dispensing, the rattle is progressively restricted in its noise-producing movement.

37. A pressurised-liquid, dispensing container as claimed in claim 36, wherein the separator is a piston with a spring urging the piston towards the liquid.

38. A pressurised-liquid, dispensing container as claimed in claim 37, wherein the spring has a free length such that as the liquid nears depletion, the spring separates from the separator and acts as the or one of the rattles.

39. A pressurised-liquid, dispensing container as claimed in claim 36, wherein the separator is a diaphragm separating propellant gas from the liquid.

40. A pressurised-liquid, dispensing container as claimed in claim 35, including two rattles progressively restricted by the restriction means, one being larger than the other, whereby the large one can become held by the restriction means before depletion with the other still movable, where the noise of the smaller one only indicates a small quantity only of contained liquid remaining.

41. A pressurised-liquid, dispensing container as claim in claim 36, including two rattles progressively restricted by the restriction means, one being larger than the other, whereby the large one can become held by the restriction means before depletion with the other still movable, where the noise of the smaller one only indicates a small quantity only of contained liquid remaining.

42. A pressurised-liquid, dispensing container as claimed in claim 37, including two rattles progressively restricted by the restriction means, one being larger than the other, whereby the large one can become held by the restriction means before depletion with the other still movable, where the noise of the smaller one only indicates a small quantity only of contained liquid remaining.

43. A pressurised-liquid, dispensing container as claimed in claim 38, including two rattles progressively restricted by the restriction means, one being larger than the other, whereby the large one can become held by the restriction means before depletion with the other still movable, where the noise of the smaller one only indicates a small quantity only of contained liquid remaining.

44. A pressurised-liquid, dispensing container as claimed in claim 39, including two rattles progressively restricted by the restriction means, one being larger than the other, whereby the large one can become held by the restriction means before depletion with the other still movable, where the noise of the smaller one only indicates a small quantity only of contained liquid remaining.

45. A pressurised-liquid, dispensing container with a cage as claimed in claim 33, wherein the cage limits the rattle from reaching one or the other or both ends of the vessel.

46. A pressurised-liquid, dispensing container as claimed in claim 32, wherein the or one of the rattles is buoyant in the liquid and the buoyant rattle is between 15% & 50% and preferably 20% & 30% of the cross-sectional dimension of the vessel.

47. A pressurised-liquid, dispensing container as claimed in claim 32, wherein:
the rattle is buoyant and
the movement restrictor is a feature of the shape of the rattle providing the rattle with length such that in an upright orientation of the container, the buoyancy of the rattle holds it against the end of the container which is upper most until the liquid approaches final depletion.

48. A pressurised-liquid, dispensing container as claimed in claim 32, wherein the or one of the rattles is non-buoyant in the liquid and the non-buoyant rattle is between 5% & 15% and preferably 7.5% & 12.5% of the cross-sectional dimension of the vessel.

49. A pressurised-liquid, dispensing container as claimed in claim 32, wherein the or one of the rattles are so arranged to produce progressively differing noise as the liquid approaches depletion.

50. A pressurised-liquid, dispensing container as claimed in claim 32, wherein the or each rattle is arranged on one end side of the or each restrictor or part thereof for indication of depletion with the opposing end of the vessel upper most.
